(19) United States
(12) Patent Application Publication
(10) Pub. No.: US 2026/0279570 A1
(43) Pub. Date: Sep. 17, 2026

(54) COLORECTAL CANCER DETECTION

(71) Applicant: Dxcover Limited, Glasgow (GB)

(72) Inventors: Matthew J. Baker, Glasgow (GB); David Palmer, Glasgow (GB); James Cameron, Glasgow (GB); Rose G. McHardy, Glasgow (GB)

(21) Appl. No.: 19/473,198

(22) PCT Filed: Apr. 11, 2024
(86) PCT No.: PCT/GB2024/050946

§ 371 Date: Oct. 7, 2025

(30) Foreign Application Priority Data
Apr. 12, 2023 (GB) 2305326.7

(51) Int. Cl. G16H 50/20; G01N 21/35; G01N 21/3577; G01N 21/552; G01N 33/49; G16B 40/10; G16B 40/20; G16H 50/70

(52) U.S. Cl. CPC G16H 50/20; G01N 21/3577; G01N 21/552; G01N 33/49; G16B 40/10; G16B 40/20; G16H 50/70; G01N 2021/3595; G01N 2201/1296

(57) ABSTRACT

A method of determining or detecting whether a subject has adenoma, comprises performing a vibrational (e.g., IR) spectroscopic analysis on a blood sample from the subject, to produce a spectroscopic signature characteristic of the blood sample, wherein said spectroscopic signature of the blood sample is analysed against representative signatures from previous subjects with and without adenoma, in order to detect whether or not the subject has adenoma, based upon the spectroscopic signature obtained from the subject

### FIELD OF THE INVENTION

The present disclosure relates to methods of detecting a pre-cancerous condition, e.g. colorectal adenoma, using spectroscopic technology.

### BACKGROUND

Early detection of cancer is vital to improve patient prognosis and reduce mortality rates. Cancer killed 10 million people in 2020, representing one of the leading causes of death worldwide. An early diagnosis can inhibit progression of the disease before the tumor proliferates to a more advanced stage. Thus, with earlier detection many patients may be cured with surgical intervention alone, meaning they would not have to endure aggressive systemic treatment like radiotherapy or chemotherapy—a reported 70% of early-stage tumors (stage I) are treated with surgery, whereas only ~13% of stage IV cancers undergo surgical resection. If the disease is not identified quickly, and the cancerous neoplasm is allowed to grow and metastasize to a distant secondary site, then the outlook for the patient is often dismal, as current therapeutic methods are rarely effective for late-stage disease.

There has recently been a plethora of research studies into liquid biopsy technologies, which have the potential to transform cancer diagnostics. Many of these tests are based upon genomic methods, which utilize genetic material such as circulating tumor DNA (ctDNA) and/or cell-free DNA (cfDNA). Remarkably, there are approximately 150,000 scientific papers that document thousands of biomarkers with apparent clinical utility, yet only 1% of known markers are routinely used in clinical applications. There are only a few commercialised liquid biopsies currently available, which are mainly targeted at single-cancer detection. For example, the SelectMDx test for prostate cancer is a urine-based test and has been successfully launched in the USA and Europe by MDxHealth. The ExoDx test (Exosome Diagnostics), also for prostate cancer, provides an individual risk score for the patient which determines whether they should be referred for biopsy. The commercial use of these single-cancer liquid biopsies evidences the potential of early detection strategies within healthcare systems.

Colorectal cancer (CRC) is the third most common cancer worldwide, with almost 2 million new cases reported in 2020. Detecting CRC early reduces mortality by enabling the removal of pre-cancerous lesions and treatment of early-stage cancers. The average 5-survival rate after diagnosis decreases from 91% in early-stage CRC, to as low as 15% for stage IV CRC. The National Institute of Health and Care Excellence (NICE) recommends screening with faecal immunochemical testing (FIT) for average-risk adults aged 50-75. Patients with elevated faecal hemoglobin (f-Hb) in their stool are referred for further investigation via colonoscopy and/or medical imaging. However, the performance of FIT is not perfect. It is highly sensitive for CRC, but false positives often arise from other causes of gastrointestinal or rectal bleeding (e.g., inflammation or haemorrhoids), resulting in f-Hb being present in the stool. Crucially, FIT cannot reliably identify pre-cancerous adenomas, e.g., advanced adenomas (AA). The sensitivity for detecting AA is only 24%, which limits the benefits of using FIT alone as a single screening tool. Furthermore, the adherence to current CRC screening programmes is extremely poor; only 43% of age-appropriate adults are compliant with FIT testing. The low screening compliance contributes to more than half of patients getting diagnosed after their disease has spread, highlighting the need for alternative detection strategies.

Perhaps the most developed technology in the CRC field is Cologuard® (Exact Sciences, USA), which is a multitarget stool test. The Cologuard® test aims to detect f-Hb in stool, and utilizes quantitative molecular assays for KRAS mutations, aberrant NDRG4 and BMP3 methylation, and β-actin, plus a hemoglobin immunoassay. For CRC detection, the sensitivity and specificity are 92% and 87%, respectively. Although, despite being greater that FIT, Cologuard still only achieves 42% AA sensitivity, suggesting the ability to detect pre-cancerous lesions could be improved.

Liquid biopsies have great potential to supplement stool-based tests, and there are various blood tests currently being developed for CRC detection. A simple blood test may be a more convenient screening option, due to the unpleasant nature of providing samples for stool-based tests which affects screening adherence. Blood testing is reportedly the preferred screening modality for CRC; more than two-thirds of patients would rather provide a blood sample than a stool sample. Therefore, a liquid biopsy could improve compliance with CRC screening.

A promising method of cancer detection using liquid biopsies is based on spectroscopic signatures obtained using attenuated total reflection infrared (IR) spectroscopy to discern cancer vs non-cancer in a subject (see WO2017/221027; Baker et al)). However, this method does not address a need in the art for the development of methods capable of detecting an earlier stage, and in particular a pre-cancerous, signature to determine pre-cancerous status in an individual, particularly in relation to colorectal cancer. Thus, the development of a robust test capable of detecting early-stage and pre-cancerous conditions such as adenomas, in relation to colorectal cancer, would be transformational within the diagnostics field.

A number of references disclose methods using infrared spectroscopy to detect cancer, such as US 2020/02000685 (Kapelushnik et al), US 2017/0285030 (Baker et al), WO 2016/097996 (Grasso et al), WO 2022/051753 (Khammanivong et al) and US 2022/0308058 (Baker et al). However, none of these documents disclose methods of pre-cancerous conditions such as adenomas.

US 2006/0269972 (Smith et al) discloses a method of detecting colorectal cancer. However, this method is based on the analysis of a stool sample, which is associated with the disadvantages highlighted above.

It is amongst the objectives of the present disclosure to develop an early detection diagnostic system that would mitigate one or more of the aforementioned disadvantages of existing screening methods.

### SUMMARY

The present is based in part on studies using vibrational (such as infrared (IR)) spectroscopy-based methods to identify pre-cancerous conditions, such as adenoma, using liquid blood samples. This method is unique as rather than searching for individual biomarkers, it probes a wide range of biological features and produces a distinctive signature which represents a significant part or whole biochemical profile of the sample. The distinctive spectroscopic signature contains molecular information from the adenoma as well as a host response, such as an immune response. The IR spectroscopy method may employ attenuated total reflection-Fourier transform infrared (ATR-FTIR) spectroscopy, which is particularly well-suited for the clinic as the methodology is minimally-invasive, cost-effective, little or no sample preparation is required, and reproducible results can be generated in a matter of minutes since analysis is rapid.

According to a first aspect, there is provided a method of determining or detecting whether a subject suspected of having a type of cancer has either adenoma or cancer irrespective of the stage of cancer, the method comprising:
performing a vibrational spectroscopic analysis on a blood sample from the subject, to produce a spectroscopic signature characteristic of the blood sample, wherein said spectroscopic signature of the blood sample is analysed against representative signatures from previous subjects with and without cancer and with and without adenoma, in order to detect whether or not the subject has either cancer or adenoma, based upon the spectroscopic signature obtained from the subject.

Advantageously, this approach allows the detection not only of a particular type of cancer, but also of early-stage and pre-cancerous conditions such as adenomas associated with that type of cancer. This may help early diagnosis of such pre-cancerous conditions, which may allow treatment at a stage earlier than would otherwise typically have been possible, thereby both reducing the scale of the treatment, and increasing dramatically the chances of survival of the subject.

Further, the method may allow a user to discern between adenoma and cancer in a subject suspected of having either adenoma or cancer. The spectroscopic signature of the blood sample may be analysed against representative signatures from previous subjects with and without cancer and with and without adenoma, in order to discern whether the subject has cancer or adenoma, based upon the spectroscopic signature obtained from the subject.

The method may allow a user to discern between different types of adenoma, adenoma, e.g. between advanced adenoma and non-advanced adenoma. The specific molecular vibrational modes and detection of peaks at specific wavenumber regions of the spectroscopic signature may also be used to discern between different types of adenoma, e.g. between advanced adenoma and non-advanced adenoma.

According to a second aspect, there is provided a method of determining or detecting whether a subject suspected of having a type of cancer has adenoma, the method comprising:
performing a vibrational spectroscopic analysis on a blood sample from the subject, to produce a spectroscopic signature characteristic of the blood sample, wherein said spectroscopic signature of the blood sample is analysed against representative signatures from previous subjects with and without adenoma, in order to detect whether or not the subject has adenoma, based upon the spectroscopic signature obtained from the subject.

Advantageously, this approach allows the detection of pre-cancerous conditions such as adenomas associated with a particular type of cancer, e.g. CRC. This may help early diagnosis of such pre-cancerous conditions, which may allow treatment at a stage earlier than would otherwise typically have been possible, thereby both reducing the scale of the treatment, and increasing dramatically the chances of survival of the subject.

The method may allow a user to discern between different types of adenoma, adenoma, e.g. between advanced adenoma and non-advanced adenoma. The specific molecular vibrational modes and detection of peaks at specific wavenumber regions of the spectroscopic signature may also be used to discern between different types of adenoma, e.g. between advanced adenoma and non-advanced adenoma.

According to a third aspect, there is provided a method of determining or detecting whether a subject suspected of having a type of cancer has either advanced adenoma or cancer irrespective of the stage of cancer, the method comprising:
performing a vibrational spectroscopic analysis on a blood sample from the subject, to produce a spectroscopic signature characteristic of the blood sample, wherein said spectroscopic signature of the blood sample is analysed against representative signatures from previous subjects with and without cancer and with and without advanced adenoma, in order to detect whether or not the subject has either cancer or advanced adenoma, based upon the spectroscopic signature obtained from the subject.

Advantageously, this approach allows the detection not only of a particular type of cancer, but also of early-stage and pre-cancerous conditions such as advanced adenomas associated with that type of cancer. This may help early diagnosis of such pre-cancerous conditions whilst excluding non-advanced adenoma to avoid unnecessary colonoscopy or treatment, which may allow targeted treatment at a stage earlier than would otherwise typically have been possible, thereby both reducing the scale of the treatment, and increasing dramatically the chances of survival of the subject.

The method may allow a user to discern between advanced adenoma and cancer in a subject suspected of having either advanced adenoma or cancer. The spectroscopic signature of the blood sample may be analysed against representative signatures from previous subjects with and without cancer and with and without advanced adenoma, in order to discern whether the subject has cancer or advanced adenoma, based upon the spectroscopic signature obtained from the subject.

Advantageously, the methods of the present disclosure may provide a more reliable way of detecting early-stage and/or pre-cancerous conditions such as adenomas associated with a particular type of cancer, e.g. CRC. This may help early diagnosis of such pre-cancerous conditions, which may allow treatment at a stage earlier than would otherwise typically have been possible, thereby both reducing the scale of the treatment, and increasing dramatically the chances of survival of the subject.

Colorectal cancer diagnosis typically relies on confirmation by colonoscopy. Thus, the methods of the present disclosure may allow one or more of the following:
Provide an additional gate-keeping test, thus increasing the reliability of the screening tests and reducing the number of patients going to colonoscopy, thus reducing costs to the medical system and avoiding unnecessary invasive procedures to the patient; and/or
Provide an additional test with improved reliability compared to conventional screening tests such as FIT, with positive results being identified as high probability patients and being fast-tracked towards colonoscopy.

The term “subject” herein refers to an individual on which detection is carried out, whereas the term “previous subjects” refers to individuals from which reference spectroscopic signatures, e.g. a database or dataset of reference spectroscopic signatures, are obtained and which can be used in subsequent comparison and/or correlation with a spectroscopic signature of the blood sample of the “subject” in order to carry out the detection, as explained below in more detail.

The term “blood” as used herein refers to whole blood or a fraction thereof. In one embodiment, the sample from the subject for the vibrational, e.g. IR, spectroscopic analysis is obtained from whole blood or a fraction thereof, such as serum or plasma. In one embodiment, the sample from the subject for the spectroscopic analysis is serum.

The term “cancer” refers to the physiological condition where cells exhibit abnormal and unregulated growth. “Cancer” as used herein, unless otherwise specified, generally refers to any type of cancer irrespective of the stage of cancer. Examples of cancer include, but are not limited to, bile duct cancer, bladder cancer, brain and central nervous system cancer, breast cancer, cervical cancer, colorectal cancer, kidney cancer, gallbladder cancer, leukaemia, liver cancer, lung cancer, stomach cancer, Hodgkin and non-Hodgkin lymphoma, melanoma, multiple myeloma, mesothelioma, osteosarcoma, oral (including lip and salivary glands) cancer, laryngeal and oro-, naso-, hypopharyngeal cancer, ovarian cancer, pancreatic cancer, prostate cancer, sarcoma, thyroid cancer, uterine and vaginal cancer, and testicular and penis cancer. Advantageously, the present disclosure, the cancer may be selected from any type of cancer capable of being associated with pre-cancerous conditions such as adenomas. As such, the cancer may be selected from a group consisting of various cancers such as colorectal cancer, gastric cancer, stomach cancer, cervical cancer, esophageal cancer, bladder cancer, breast cancer, liver cancer, lung cancer, prostate cancer and vaginal cancer.

The term “adenoma” herein refers to a benign tumour which is generally considered as a pre-cancerous condition. Adenoma may comprise several sub-types which may be categorised as “advanced adenoma” (AA) or “non-advanced adenoma” (NAA). Advanced adenoma may comprise or may consist of adenoma classed as either (i) high-grade dysplasia or a villous growth pattern (any size) or (ii) adenomas/serrated lesions >10 mm in size. Non-advanced adenoma may comprise or may consist of adenoma being <10 mm in size.

The type of cancer may be colorectal cancer (CRC) and/or the type of adenoma may be colorectal adenoma.

The subject may be suspected of having colorectal cancer.

Thus, in an embodiment of the first aspect, there is provided a method of determining or detecting whether a subject suspected of having colorectal cancer has either colorectal adenoma or colorectal cancer irrespective of the stage of cancer, the method comprising:
performing a vibrational spectroscopic analysis on a blood sample from the subject, to produce a spectroscopic signature characteristic of the blood sample, wherein said spectroscopic signature of the blood sample is analysed against representative signatures from previous subjects with and without colorectal cancer and with and without colorectal adenoma, in order to detect whether or not the subject has either colorectal adenoma or colorectal cancer, based upon the spectroscopic signature obtained from the subject.

In an embodiment of the second aspect, there is provided a method of determining or detecting whether a subject suspected of having colorectal cancer has colorectal adenoma, the method comprising:
performing a vibrational spectroscopic analysis on a blood sample from the subject, to produce a spectroscopic signature characteristic of the blood sample, wherein said spectroscopic signature of the blood sample is analysed against representative signatures from previous subjects with and without colorectal adenoma, in order to detect whether or not the subject has colorectal adenoma, based upon the spectroscopic signature obtained from the subject.

In an embodiment of the third aspect, there is provided a method of determining or detecting whether a subject suspected of having colorectal cancer has either advanced colorectal adenoma or colorectal cancer irrespective of the stage of cancer, the method comprising:
performing a vibrational spectroscopic analysis on a blood sample from the subject, to produce a spectroscopic signature characteristic of the blood sample, wherein said spectroscopic signature of the blood sample is analysed against representative signatures from previous subjects with and without colorectal cancer and with and without advanced colorectal adenoma, in order to detect whether or not the subject has either colorectal cancer or advanced colorectal adenoma, based upon the spectroscopic signature obtained from the subject.

Typically, the vibrational spectroscopic analysis may comprise or may consist of IR spectroscopic analysis comprising wavelengths between 400-4000 cm⁻¹. The IR spectroscopic analysis may comprise or may consist of ATR-IR spectroscopic analysis.

The previous subjects with a type of cancer, e.g. colorectal cancer, may comprise subjects with different stages of cancer, e.g. of colorectal cancer.

As used herein, “stage of cancer” refers to the size of the tumour and indication of the spread of the cancer from the tissue where the cancer has originated from, which enables clinicians to assess cancer progression. Most cancers that involve a tumour can be categorised into four stages, according to the Overall Stage Grouping, also known as Roman Numeral Staging. Stage I refers to when the cancer is small and localised within the organ the cancer started in. Stage II refers to when the cancer has grown and is larger than stage I but has not spread to surrounding tissues. In some types of cancers, stage II may include instances where the cancer has spread to nearby lymph nodes. Early-stage cancer typically refers to stage I or stage II cancers. Stage III refers to when the cancer is larger and may have spread to the surrounding tissues, such as nearby tissues and/or lymph nodes. Stage IV refers to when the cancer has spread to other organs or has advanced to a substantial volume for organ confined tumours (e.g. brain cancer) (also referred to as advanced or metastatic cancer). In an embodiment the methods as described herein are particularly suited to the detection of early-stage cancers, typically stages I-III, or I-II. In another embodiment the methods as described herein are particularly suited to the detection of cancers irrespective of the stage of cancer, and/or the methods as described herein are particularly suited to the detection of cancers in any of stages I-IV.

As used herein, the term “detecting” is used broadly and may be used in terms of facilitating with a diagnosis and/or prognosis of a subject. The detection of cancer in a subject may comprise conducting an analysis against representative signatures obtained from (the) previous subjects. This may involve comparing and/or correlating the spectroscopic signature of the blood sample (or component thereof) to one or more reference spectroscopic signatures previously obtained by statistical analysis from (the) previous subjects with and without cancer and with and without adenoma. Comparing and/or correlating may be carried out using a suitable computational process or program. The method may comprise comparing and/or correlating, using a machine learning or deep learning process.

The machine learning or deep learning process may be or may comprise a neural network. The neural network may be or may comprise a convolutional neural network or a recurrent neural network. The convolutional neural network may comprise one or more of: at least one 1-dimensional convolutional layer, at least one down sampling layer and/or at least one batch normalization layer. The recurrent neural network may comprise one or more long-short term memory (LSTM) layers. Other exemplary algorithms which may be used include: (oblique) random forest with partial least squares (PLS), logistic regression, support vector machines or other machine learning algorithms at each node in each decision tree; other linear models such as LDA. The machine learning or deep learning models may be single models or ensembles of multiple models. Ensemble models may be built on different samples of the data using one or more of bootstrap aggregating (“bagging”) or random sampling.

The data may be, may comprise, may be derived from, or may be representative of directly measurable spectroscopic data, comprising or resulting from spectral measurements taken from the blood sample. The data may be or may comprise measurements, which are taken across the defined wavelength region, or may be or may comprise measurements obtained from two or more sub-regions, as will be discussed further herein.

According to an example of the present disclosure there is provided a computer readable medium carrying a computer program comprising computer readable instructions configured to cause a computer to carry out a method as described herein.

According to an example of the present disclosure there is provided a computer apparatus, such as embodied within, or connected to a spectrometer, comprising or configured to access: a memory storing processor readable instructions, and a processor arranged to read and execute instructions stored in said memory, wherein said processor readable instructions comprise instructions arranged to control the computer to carry out a method as described herein.

Examples of the subject matter and the functional operations described in this specification can be implemented in digital electronic circuitry, in tangibly-embodied computer software or firmware, in computer hardware, including the structures disclosed herein and their structural equivalents, or in combinations of one or more of them. Examples of the subject matter described in this specification can be implemented as one or more computer programs, i.e., one or more modules of computer program instructions encoded on a tangible non-transitory program carrier for execution by, or to control the operation of, data processing apparatus. Alternatively, or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “data processing apparatus” encompasses all kinds of apparatus, devices, and machines for processing data, including by way of example a programmable processor, a computer, or multiple processors or computers. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a standalone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, subprograms, or portions of code. A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Computers suitable for the execution of a computer program include, by way of example, can be based on general or special purpose microprocessors or both, or any other kind of central processing unit. Generally, a central processing unit will receive instructions and data from a read only memory or a random-access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, examples of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the internet.

The computing system can include clients and servers. A client and server are generally remote from each other and typically interact through a communication network. The relationship of client and server arises by virtue of computer programs running on the respective computers and having a client-server relationship to each other.

References are made herein to condition parameter curves, but it will be appreciated that such references are to the evolution of the degradation parameter and may comprise linear and/or arcuate sections such as sections that can be described by polynomial, exponential, power and other functions, and may be smoothly or gradually varying or may comprise sharp, angular transitions between regions.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Typically, the vibrational spectroscopic analysis may comprise or may consist of IR spectroscopic analysis comprising wavelengths between 400-4000 cm-1. The IR spectroscopic analysis may comprise or may consist of ATR-IR spectroscopic analysis.

However, it will be understood that other vibrational spectroscopic analysis techniques may be employed, such as Raman spectroscopy or the like.

The methods of the present disclosure typically use infrared (IR) spectroscopic analysis. Various IR techniques known in the art may be employed in the methods of this disclosure. One method may use Fourier transform IR (FTIR) spectroscopic analysis. In FTIR, the IR spectra may be collected in the region of 400-4000 wavenumbers (cm-1). Generally, the IR spectra may have a resolution of 10 cm⁻¹ or less, 5 cm⁻¹ or less, or approximately 4 cm⁻¹. The FTIR spectroscopic analysis may employ at least 10 scans, at least 15, or at least 30 scans. The FTIR spectroscopic analysis may employ at most 100 scans, at most 50 scans, or at most 40 scans. For example, 16 scans may be used. The scans may be co-added. As will be appreciated by the skilled person, the number of scans may be selected to optimize data content and data-acquisition time.

Prior to spectroscopic analysis, a background spectrum may be obtained. Such background spectra may provide correction for a background environment. For example, the background spectrum of air may be obtained to provide an atmospheric correction. Alternatively, or additionally, the background spectrum of a solution may be obtained, e.g. an aqueous solution such as phosphate buffered saline (PBS). In one embodiment, a background spectrum is obtained in order to provide for correction for a background environment. In one embodiment, the spectroscopic analysis may further comprise normalisation (e.g. standard normal variate), noise reduction (e.g. principal component analysis (PCA)-based) and derivatisation (e.g. 1st or 2nd) as pre-processing steps and/or Fourier transform IR spectroscopic analysis.

The claimed methods use vibrational spectroscopic analysis, typically IR spectroscopic analysis, such as Attenuated Total Reflection (ATR)-IR spectroscopic analysis, using blood samples from subjects. In some embodiments, the spectroscopic analysis used to obtain spectroscopic signature characteristic of the blood sample may be ATR-FTIR.

During a typical ATR-IR spectroscopic analysis, the blood sample of a subject may be loaded onto an internal reflection element (IRE) and IR light may travel through the IRE, and reflect (e.g. via total internal reflection) at least once off an internal surface of the IRE that is in contact with the sample. Such reflection may form an “evanescent wave” which penetrates into the blood sample to an extent depending on the wavelength of light, the angle of incidence and the indices of refraction for the IRE and the blood sample itself. The depth of penetration and path of reflected light can be altered by varying the angle of incidence and/or wavelength of incident light. The beam may be received by an IR detector as it exits the internal reflection element. The IRE is generally an optical material with a higher refractive index than the blood sample to enable the evanescent wave effect.

The IR spectroscopic signature characteristic of the blood sample (which may be referred to as the signature or fingerprint region) may typically be part or all of the relevant IR spectrum between 400 to 4000 cm⁻¹.

The spectroscopic signature characteristic of the blood sample may be compared to a database of representative signatures previously obtained from samples from previous subjects with cancer or with adenoma or healthy subjects, in order to detect, through comparison of the respective signatures, whether or not the subject has cancer or adenoma. Such comparison may be carried out using pattern recognition software and/or machine learning analysis known in the art and/or as described herein.

The spectroscopic signature characteristic of the blood sample may be compared to a database of representative signatures previously obtained from samples from previous subjects with adenoma or healthy subjects, in order to detect, through comparison of the respective signatures, whether or not the subject has adenoma. Such comparison may be carried out using pattern recognition software and/or machine learning analysis known in the art and/or as described herein.

In one embodiment, the analysis against representative signatures from previous subjects comprises applying a trained model to the spectroscopic signature. The trained model may comprise a trained machine learning model, and optionally a neural network, a supporting vector machine (SVM) or a random forest (RF) decision tree. The trained model may comprise or function as a classifier by applying the or a probability threshold to a probability value output by the trained model. The method may additionally comprise selecting and/or varying the probability threshold thereby selecting and/or varying the specificity and/or sensitivity of the spectroscopic signature analysis. Further, the method may involve selecting the probability threshold based on a receiver operating characteristic (ROC) curve for the trained model, and/or selecting the trained model from a set of trained models based on ROCs for the set of trained models, for example thereby to obtain a desired specificity and/or sensitivity.

The term “sensitivity” is herein understood as the proportion of diseased subjects who are correctly identified as “positive” by a test. Thus, sensitivity can be defined as the percentage of true positives, as predicted by the test. A test with 100% sensitivity would correctly detect all patients who have a given disease. In other words, high sensitivity means a low occurrence of false negatives.

The term “specificity” is herein understood as the proportion of non-diseased subjects who are correctly identified as “negative” by a test. Thus, specificity can be defined as the percentage of true negatives, as predicted by the test. A test with 100% specificity would correctly detect all patients who do not have a given disease. In other words, high specificity means a low occurrence of false positives.

An advantageous feature of the methods described herein is the ability to detect (colorectal) adenoma, or (colorectal) adenoma and/or (colorectal) cancer, in a patient. Advantageously, the methods are capable of detecting early-stage cancer and/or pre-cancerous conditions, as an early medical intervention may increase the likelihood of patient survival. For this to be possible as a first-line diagnostic test, high sensitivity of the test is essential in order to ensure that all patients with adenoma (and optionally cancer) are correctly identified. Existing pre-cancerous diagnostic tests, such as FIT, are not sensitive enough to provide a reliable methodology. The spectroscopic liquid biopsy methods as disclosed herein differ from other tests as the probability threshold of the machine learning analysis can be adjusted to maximise either the sensitivity or specificity depending on clinical requirements. In one embodiment, there is provided a method comprising a spectroscopic liquid biopsy with machine learning analysis, wherein the probability threshold of the analysis is adjusted to maximise the sensitivity for detection of (colorectal) adenoma, using blood samples from patients.

In some application of diagnostic analysis, such as when stratifying subjects with either adenoma or cancer from healthy subjects in a triage setting, classifiers with a high sensitivity and modest to low specificity may be more desirable than models with low sensitivity and high specificity. In other diagnostic applications, such as for general adenoma/cancer detection with a population level screening test, classifiers with high specificity and modest to low sensitivity may be more desirable. The desired level of performance is generally selected based upon a trade-off that must be made between the number of false positive and false negatives that can each be tolerated for the particular diagnostic applications. Such trade-offs generally depend on the medical consequences of an error. In one embodiment, the method as described herein comprises adjusting the probability threshold of the machine learning algorithm to determine the sensitivity and/or specificity of the spectroscopic analysis.

A high sensitivity or specificity typically refers to a value greater than 70%. A modest sensitivity or specificity may typically be greater than 50% but less than 70%. In one embodiment, the sensitivity of the analysis is greater than 70% and specificity of the analysis is greater than 40%. In another embodiment, the sensitivity of the analysis is greater than 40% and the specificity of the analysis is greater than 70%. In one embodiment, the sensitivity of the analysis is at least 80% and specificity of analysis is at least 40%. In one embodiment, the sensitivity of the analysis is at least 40% and specificity of analysis is at least 80%. In certain embodiments, the sensitivity is at least 80% and specificity is at least 40%, 45%, 50%, 55%, 60%, 65%, 70% or 75%. In certain embodiments, the specificity is at least 80% and sensitivity is at least 40%, 45%, 50%, 55%, 60%, 65%, 70% or 75%.

In one embodiment, the spectroscopic analysis of the present disclosure comprises conducting the spectroscopic analysis with high sensitivity to identify patients with adenoma, or with adenoma and/or cancer, and subsequently conducting the spectroscopic analysis with high specificity to identify the patients without adenoma, or without adenoma or cancer.

The present disclosure also provides a method of using the spectroscopic signature of the blood sample comprising specific molecular vibrational modes and/or detection of peaks at specific wavenumber regions to detect adenoma, or adenomas and/or cancer. The specific molecular vibrational modes and detection of peaks at specific wavenumber regions of the spectroscopic signature may also be used to discern between adenoma and cancer. The specific molecular vibrational modes and detection of peaks at specific wavenumber regions of the spectroscopic signature may also be used to discern between different types of adenoma, e.g. between advanced adenoma and non-advanced adenoma. Detection of peaks at specific wavenumber regions are associated with molecular vibrational modes that may be used to identify the type of condition of interest in the subject, e.g. adenoma or cancer, irrespective of the stage of cancer.

The analysing against representative signatures from previous subjects may comprise applying a trained model to the spectroscopic signature.

The trained model may comprise a trained machine learning model, optionally a neural network, a support vector machine (SVM) or a random forest (RF) decision tree. Alternatively, or additionally, any other suitable model, or model features, may be used for example ORF-PLS, ORF-SVM and/or bagging models, shrinkage discriminant analysis or distance weighed discrimination (DWD) linear models.

The trained model may comprise or functions as a classifier by applying the, or a, probability threshold to a probability value output by the trained model.

The method may further comprise selecting and/or varying the probability threshold thereby selecting and/or varying the specificity and/or sensitivity of the spectroscopic signature analysis.

The method may further comprise selecting the probability threshold based on a receiver operating characteristic (ROC) curve for the trained model, and/or selecting the trained model from a set of trained models based on ROCs for the set of trained models, for example thereby to obtain a desired specificity and/or sensitivity.

One method of detecting presence of adenoma, or adenoma and/or cancer in a subject, and optionally if the subject has adenoma and/or cancer, whether the subject has adenoma or cancer, using the method as described in the present disclosure is through the analysis of molecular vibrational modes obtained using ATR-IR analysis of the subject's blood sample or similar methods.

In one embodiment, the method of detecting presence of adenoma, or adenoma and/or cancer, in a subject, comprises analysis of molecular vibrational modes selected from: N—H (in-plane) bend/deformation, C—N stretch, C—H stretch/deformation, CH₂ stretch, C—O stretch, C—C stretch, C—OH deformation, CH₂ wagging, C═O stretch, asymmetric PO₂ stretch and/or symmetric PO₂ stretch.

In one embodiment, the method of detecting whether or not the subject has adenoma, or adenoma and/or cancer, is based on the analysis of one or more vibration modes of the spectroscopic signature selected from: C—O stretch, C—C stretch, C—H deformation, N—H bend, C—N stretch and/or C═O stretch. In another embodiment, the method of detecting whether or not the subject has adenoma, or adenoma and/or cancer, is based on the analysis of vibration modes of the spectroscopic signature comprising C—O stretch, C—C stretch, C—H deformation, N—H bend, C—N stretch and C═O stretch.

In one embodiment, the method of detecting colorectal adenoma, or colorectal adenoma and/or colorectal cancer, in a subject is based on the analysis of one or more vibrational modes of the spectroscopic signature selected from: C═O stretch, C—N stretch, N—H bend, C—O stretch, C—C stretch and/or C—H deformation. In another embodiment, the method of detecting colorectal adenoma, or colorectal adenoma and/or colorectal cancer in a subject is based on the analysis of vibrational modes of the spectroscopic signature comprising C═O stretch, C—N stretch, N—H bend, C—O stretch, C—C stretch and C—H deformation.

An alternative spectroscopic feature that may be used to detect presence of adenoma, or adenoma and/or cancer, in a subject, is through identification of peaks at distinct wavenumber regions in the spectroscopic signature of the sample from the subject.

In one embodiment, the method for detecting whether or not the subject has adenoma, or adenoma and/or cancer, and optionally if the subject has adenoma and/or cancer, whether the subject has adenoma or cancer, is based on the identification of peaks in the spectroscopic signature of the sample at one or more wavenumber regions within 400-4000 cm⁻¹ or a portion or portions thereof. In an alternative embodiment, the method for detecting whether or not the subject has adenoma, or adenoma and/or cancer, and optionally if the subject has adenoma and/or cancer, whether the subject has adenoma or cancer, is based on the identification of peaks in the spectroscopic signature of the sample at one or more wavenumber regions within 1000-3700 cm⁻¹ or a portion or portions thereof.

In one embodiment, the method of detecting whether or not the subject has adenoma, or adenoma and/or cancer is based on identification of peaks at one or more wavenumbers: 1163 cm⁻¹, 1578 cm⁻¹ and/or 1682 cm⁻¹. In another embodiment, the method of detecting whether or not the subject has adenoma, or adenoma and/or cancer, is based on identification of peaks at 1163 cm⁻¹, 1578 cm⁻¹ and 1682 cm⁻¹.

In one embodiment, the method of detecting whether or not the subject has colorectal adenoma, or colorectal adenoma and/or colorectal cancer, is based on identification of peaks at one or more wavenumbers: 1682 cm⁻¹, 1575 cm⁻¹ and/or 1165 cm⁻¹. In another embodiment, the method of detecting whether or not the subject has colorectal adenoma, or colorectal adenoma and/or colorectal cancer, is based on identification of peaks at 1682 cm⁻¹, 1575 cm⁻¹ and 1165 cm⁻¹.

In an embodiment, any of the abovementioned wavenumber regions may vary by ±100 cm⁻¹, ±50 cm⁻¹, ±40 cm⁻¹, ±30 cm⁻¹, ±20 cm⁻¹ and/or ±10 cm⁻¹.

The spectroscopic signature may be correlated with a favourable or unfavourable diagnosis and/or prognosis based on a predictive model developed by “training” (e.g. via pattern recognition and/or machine learning algorithms) a database of pre-correlated analyses. In order to train a model, we provide known cancer and known non-cancer samples from the appropriate patient population in order to identify the signature that can enable discrimination. Correlating the analytical results with a favourable or unfavourable diagnosis and/or prognosis may be performed manually (e.g. by a clinician or other suitable analyst) or automatically (e.g. by computational means). Correlations may be established qualitatively (e.g. via a comparison of graphical traces or signatures) or quantitatively (e.g. by reference to predetermined threshold values or statistical limits). Correlating the analytical results may be performed using a predictive model, optionally as defined herein, which may have been developed by “training” a database of pre-correlated assays and/or analyses.

In one embodiment, a computer program product may be provided comprising computer-readable instructions that are executable to perform the method as described herein. A trained machine learning model may be configured to receive an input comprising a spectroscopic signature of an IR spectroscopic analysis comprising wavelengths between 400-4000 cm⁻¹ performed on a blood sample from the subject and to provide an output representative of whether or not the subject has adenoma, or adenoma and/or cancer. In one embodiment, the method of training a machine learning model may comprise receiving a plurality of data sets representing spectroscopic signatures for a plurality of subjects obtained from IR spectroscopic analysis comprising wavelengths between 400-4000 cm⁻¹ on blood samples from the subjects; receiving adenoma, or adenoma and/or cancer, data indicating for at least some of the subjects whether the subject has adenoma, or adenoma and/or cancer; and training the model to determine a probability of whether a patient has adenoma, or adenoma and/or cancer, based on a spectroscopic signature for the patient, wherein some of the subjects have adenoma, or adenoma and/or cancer. The training of the model may also comprise tuning at least one parameter of the model to optimise the area under the ROC curve and/or to provide a desired sensitivity and/or specificity for a determination as to whether a patient has adenoma, or adenoma and/or cancer.

The detection method of the present disclosure provides a method of analysing macromolecules in a minute volume of patient serum through IR spectroscopic analysis and machine learning algorithms. As the analysis only requires a small volume of blood, it may be feasible to integrate this rapid liquid biopsy spectroscopic test with other existing blood-based tests without disrupting clinical practice, such as taking a small aliquot of blood from a routine blood test. In one embodiment, the small aliquot taken from the blood sample for IR analysis may typically be less than 1 mL. In alternative embodiments, the small aliquot taken from the blood sample for IR analysis may be less than 100 μL, 80 μL, 60 μL, 40 μL or 10 μL. In this manner, the bulk (typically over 80%, 90%, 95%, or 99%) of the original sample is still available for subsequent analysis.

The methods of the present disclosure may therefore permit effective triage of patients, by expediting further assessment for patients more at risk while excluding a diagnosis, e.g. colonoscopy, in others. In addition, it may identify pre-cancerous conditions, e.g. adenoma, and optionally sub-types of adenoma, in asymptomatic patients or patients with non-specific symptoms if offered in conjunction with other blood based diagnostic assays or routine blood tests. Optionally, the methods may further be effective irrespective of the age and/or the sex of the subjects.

In one embodiment, the methods of the present disclosure may be conducted as a standalone test or optionally as an additive test in combination with other blood-based tests.

In one embodiment, the methods as disclosed herein may conducted in combination with one or more other blood-based tests using a single blood draw, wherein a first aliquot is used to conduct the IR spectroscopic analysis as described herein and a second aliquot comprising the remainder of the blood sample, or a portion thereof, is used to conduct one or more other blood-based tests. In one embodiment, the remainder of the blood draw may be used to conduct standard routine blood tests or other liquid biopsy based diagnostic tests. In an alternative embodiment, the further analysis may comprise conducting a follow-up test, such as liquid biopsy sequencing assays (e.g., NGS-based ctDNA tests, methylation tests, biomarker tests). The single blood draw may be aliquoted into separate containers or receptacles, or the single blood draw may be provided into a single container or receptacle.

In a further aspect, there is provided a computer program product comprising computer readable instructions that are executable to perform a method as claimed or described herein.

In another aspect, which may be provided independently, there is provided a trained model configured to receive an input comprising a spectroscopic signature of an ATR-IR spectroscopic analysis comprising wavelengths between 400-4000 cm⁻¹ performed on a blood sample from the subject and to provide an output representative of whether or not the subject has adenoma, or adenoma and/or cancer.

In another aspect, which may be provided independently, there is provided a method of training a model comprising receiving a plurality of data sets representing spectroscopic signatures for a plurality of subjects obtained from ATR-IR spectroscopic analysis comprising wavelengths between 400-4000 cm⁻¹ on blood samples from the subjects; receiving adenoma, or adenoma and/or cancer, data indicating for at least some of the subjects whether the subject has adenoma, or adenoma and/or cancer; and training the model to determine a probability of whether a patient has adenoma, or adenoma and/or cancer, based on a spectroscopic signature for the patient, wherein some of the subjects have adenoma, or adenoma and/or cancer.

The training of the model may comprise tuning at least one parameter of the model to optimise the area under the ROC curve and/or to provide a desired sensitivity and/or specificity for a determination as to whether a patient has adenoma, or adenoma and/or cancer.

Features in any one aspect may be provided as features in any one or more other aspects. For example, any of method, computer program product, model or apparatus features may be provided as any one or more other of method, computer program product, model or apparatus features.

### BRIEF DESCRIPTION OF DRAWINGS

The present disclosure will now be further described by way of example and with reference to the Figures, which show:

FIG. 1: Schematic breakdown of the full patient cohort.

FIG. 2: Results showing the mean receiver operating characteristic curve for (a—pink) colorectal cancer (CRC) versus non-cancer (NC), (b—purple) Adenoma (A) v NC, and (c—blue) CRC+A v NC, illustrating the trade-off between sensitivity (Sens) and specificity (Spec). AUC denotes the area under the curve.

FIG. 3: Detection rates for each colorectal cancer (CRC) stage, and the overall CRC and advanced adenoma (AA) sensitivity from the CRC+A v NC model, when specificity is fixed at 90%. AA sensitivity does not consider the ‘non-advanced’ adenomas (n=7); overall adenoma sensitivity is 58%.

### DETAILED DESCRIPTION

In the present disclosure, reference is made to a number of terms, which have the meanings provided below, unless a context indicates to the contrary.

The term “comprising” or variants thereof is to be understood herein to imply the inclusion of a stated element, integer or step, or group of elements, integers or steps, but not the exclusion of any other element, integer or step, or group of elements, integers or steps.

The term “consisting” or variants thereof is to be understood to imply the inclusion of a stated element, integer or step, or group of elements, integers or steps, and the exclusion of any other element, integer or step or group of elements, integers or steps.

The term “about” herein, when qualifying a number or value, is used to refer to values that lie within +5% of the value specified. For example, if a temperature is specified to be about 5 to about 13° C., temperatures of 4.75 to 13.65° C. are included.

### Methods

### Patient Sample Cohort Selection

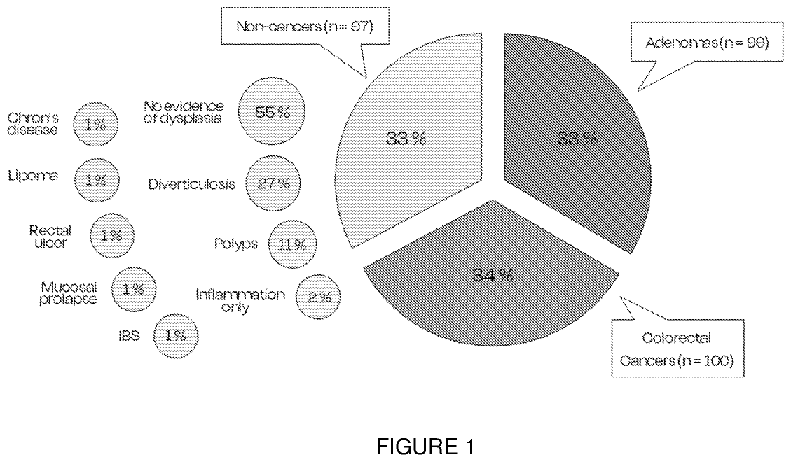

The patient samples included in this study were sourced from Tayside Biorepository (Dundee, UK). Ethical approval was granted (#22/ES/0041-TR628) for sample collection. Blood samples were taken from the patients prior to scheduled colonoscopy, before surgical resection and any anti-cancer therapies. The full cohort consisted of 296 patients: 100 CRC, 99 adenoma (A) and 97 colonoscopy screening controls with a non-cancer (NC) diagnosis. A schematic breakdown of the full patient cohort is shown in FIG. 1.

All cancer samples were collected from patients with a histopathological confirmed CRC diagnosis. The adenoma samples were categorized as advanced adenoma (AA) if they were classed as either (i) carcinoma in situ or high-grade dysplasia or a villous growth pattern (any size) or (ii) adenomas/serrated lesions >10 mm in size. Adenoma samples which were <10 mm in size were classed as ‘non-advanced’.

The NC group consisted of other non-malignant conditions such as small polyps, diverticulosis, and inflammation.

Blood samples were obtained with venipuncture using red-topped BD Vacutainer™ serum collection tubes, then anonymized. Serum was extracted via centrifugation and stored in a −80° C. freezer. Non-identifiable clinical and demographic data were obtained in-line with the biobank data control procedures.

### Patient Sample Analysis

Serum aliquots were removed from frozen storage (−80° C.) and thawed for up to 30 minutes at room temperature (18-25° C.) and inverted three times to ensure mixing before use. Each patient sample was prepared for analysis by pipetting 3 μL of serum onto each of the three sample wells of a Dxcover® Sample Slide (Dxcover Ltd, UK). Prepared slides were placed in an incubator (Thermo Scientific™ Heratherm™, USA) at 35° C. for at least 10 minutes to create dried serum films. Each dried sample slide was then placed into a Dxcover® Autosampler (Dxcover Ltd, UK) coupled with a PerkinElmer® Spectrum Two™ FTIR spectrometer (PerkinElmer® Inc., USA), and the Dxcover® Platform Software (Dxcover Ltd., UK) automated spectral data acquisition. Three spectra were collected for each sample well, resulting in nine replicates per patient.

### Data Analysis

Machine learning models were developed to build a diagnostic algorithm from the known patient population and enable disease predictions for unknown samples in the test sets. Three different classifications were examined in this study:

$$CRC v NC ; \qquad ( a )$$

$$A v NC ; and \qquad ( b )$$

$$CRC + A v NC . \qquad ( c )$$

A nested cross-validation (CV) strategy was used to develop the models to reduce sampling bias. In this approach, patients were randomly split into training and test sets with a 70:30 split, repeated 51 times. Model hyper-parameters were tuned on the training set (70%), which was used to make predictions for the spectra in the test set (30%). Since each patient sample provides nine spectra, the final diagnosis was taken as the consensus prediction (maximum vote) from all nine spectra. Patient samples were reported as positive or negative according to the diagnostic algorithm results. Spectra from individual patients were not allowed to be present in both the training and test sets for a given resample. The classification metrics obtained from all 51 outer CV iterations were aggregated, and the mean values have been reported. For each patient, the predictions from all the test sets in which that patient is present were collected and the majority vote taken as the overall test set prediction for that patient. From this, an overall detection rate (sensitivity) was calculated as the ratio of correct predictions to total number of predictions, which is reported for cancer and adenomas, and for the CRC patients when split by stage, when specificity is fixed at 90%.

### Results

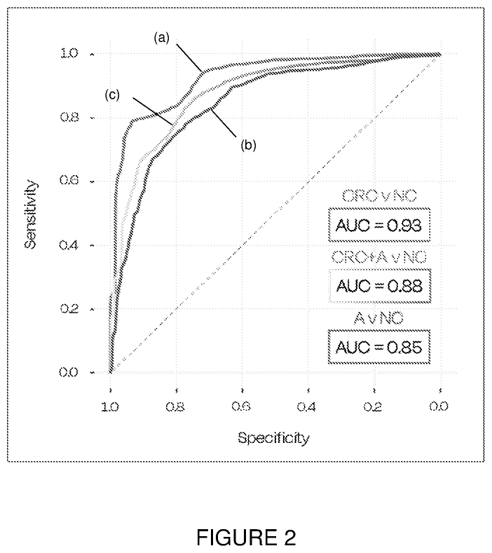

The mean receiver operating characteristic (ROC) curves for each classification are illustrated in FIG. 2.

Firstly, the CRC v NC model reported an area under the curve (AUC) value of 0.93, which indicates the test has excellent discriminating ability. The A v NC model has an AUC of 0.85, which is still a promising result considering adenomas are a pre-cancerous condition and are known to be difficult to detect with currently available screening methodologies. As expected, the CRC+A v NC ROC curve sits in-between the other two curves, as we are combining the cancers and adenomas into one group. It is envisaged that this may arguably represent the most useful classifier since an effective screening test should identify both CRC and patients with pre-cancerous lesions for rapid referral to colonoscopy. Thus, an AUC of 0.88 highlights the potential of this liquid biopsy as a CRC screening tool.

However, it will be appreciated that other classifications may also be useful. For example, in other embodiments, only certain types of adenoma (e.g., AA) may be grouped with CRC, for example in a AA+CRC v NC analysis represented by an associated AA+CRC v NC curve, whilst non-advanced adenoma (e.g. <10 mm in size) may be grouped with the NC group. Ultimately, as the spectroscopic signature of a sub-group is characteristic of that sub-group and can be used by a machine learning model and associated diagnostic algorithm, any selection of adenoma sub-groups can be considered depending on the ultimate diagnostics outcome desired by a user or practitioner.

In the results shown in FIG. 2, when specificity was fixed at 90%, the CRC sensitivity for the CRC v NC model was 80%. This model accurately predicted 83% of stage 1, 73% of stage II, 76% of stage III and 100% of stage IV cancers. For the A v NC model, the sensitivity for all adenomas was 58%.

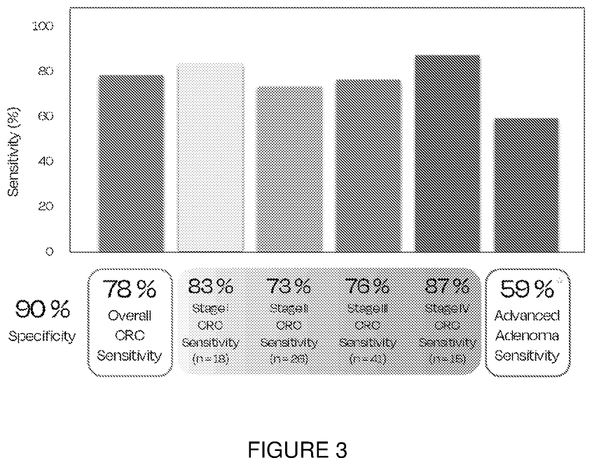

FIG. 3 illustrates the sensitivities for the CRC+A v NC model at 90% specificity, which shows very similar results to the CRC v NC model. However, the sensitivity for CRC sensitivity was 78% in this case, and 87% of stage IV cancers were detected. When the stages are combined, 77% of early-stage (I/II) and 79% of late-stage (III/IV) tumors were successfully identified. Moreover, 58% of all adenomas were detected with this model, 7 of which were classed as ‘non-advanced’ (e.g., <10 mm in size). Notably, 54 out of 92 (59%) AA patients were predicted correctly.

### Discussion

The findings from this study suggests the methods of the present disclosure have significant potential to be employed as a screening tool for CRC, including pre-cancerous conditions such as adenoma. The classification metrics can be fine-tuned depending upon the requirements of the diagnostic pathway and healthcare system. For example, sensitivity (or specificity) can be augmented while ensuring the specificity (or sensitivity) is at an approximate value. Many of the liquid biopsy technologies for CRC that are currently under development report their maximum sensitivities when specificity is fixed at 90%. This is likely due to the minimum performance levels set by the Centers for Medicare & Medicaid Services (CMS) for coverage of CRC tests (74% sensitivity/90% specificity). For the CRC+A v NC model—which is likely to be the most appropriate dataset for a screening setting—the overall CRC sensitivity was 78% (at 90% specificity), which surpasses the CMS targets. When split by stage, the present approach successfully detected 83% of stage I and 73% of stage II tumors, showing great potential for early-stage CRC detection. Additionally, 76% and 87% of stage III and IV were identified. Furthermore, 59% of AA patients were predicted correctly by the CRC+A v NC model. This is believed to be the highest sensitivity reported to date for AA from blood-based CRC tests.

It will be understood that the present embodiments are provided by way of example only, and that various modifications can be made to the present embodiments without departing from the scope of the invention.

1. A method of determining or detecting whether a subject suspected of having a type of cancer has either adenoma or cancer irrespective of the stage of cancer, the method comprising:
performing an infrared (IR) spectroscopic analysis on a blood sample from the subject, to produce a spectroscopic signature characteristic of the blood sample, wherein said spectroscopic signature of the blood sample is analysed against representative signatures from previous subjects with and without cancer and with and without adenoma, in order to detect whether or not the subject has either cancer or adenoma, based upon the spectroscopic signature obtained from the subject.

2. A method of determining or detecting whether a subject suspected of having a type of cancer has adenoma, the method comprising:
performing an infrared (IR) spectroscopic analysis on a blood sample from the subject, to produce a spectroscopic signature characteristic of the blood sample, wherein said spectroscopic signature of the blood sample is analysed against representative signatures from previous subjects with and without adenoma, in order to detect whether or not the subject has adenoma, based upon the spectroscopic signature obtained from the subject.

3. A method of determining or detecting whether a subject suspected of having a type of cancer has either advanced adenoma or cancer irrespective of the stage of cancer, the method comprising:
performing an infrared (IR) spectroscopic analysis on a blood sample from the subject, to produce a spectroscopic signature characteristic of the blood sample, wherein said spectroscopic signature of the blood sample is analysed against representative signatures from previous subjects with and without cancer and with and without advanced adenoma, in order to detect whether or not the subject has either cancer or advanced adenoma, based upon the spectroscopic signature obtained from the subject.

4. The method according to claim 1, wherein the IR spectroscopic analysis comprises wavelengths between 400-4000 cm⁻¹.

5. The method according to claim 4, wherein the IR spectroscopic analysis comprises ATR-IR spectroscopic analysis.

6. The method according to claim 1, wherein the type of cancer is colorectal cancer (CRC) and/or wherein the type of adenoma is colorectal adenoma.

7. The method according to claim 1, wherein the analysing against representative signatures from previous subjects comprises applying a trained model to the spectroscopic signature.

8. The method according to claim 7, wherein the trained model comprises a trained machine learning model, optionally a neural network, a support vector machine (SVM) or a random forest (RF) decision tree.

9. The method according to claim 7, wherein the trained model comprises or functions as a classifier by applying the er a probability threshold to a probability value output by the trained model.

10. The method according to claim 9, further comprising selecting and/or varying the probability threshold thereby selecting and/or varying the specificity and/or sensitivity of the spectroscopic signature analysis.

11. The method according to claim 9, further comprising selecting the probability threshold based on a receiver operating characteristic (ROC) curve for the trained model, and/or selecting the trained model from a set of trained models based on ROCs for the set of trained models, for example thereby to obtain a desired specificity and/or sensitivity.

12. The method according to claim 1, wherein the spectroscopic analysis comprises detection of vibrational mode(s) and/or wavenumber regions.

13. The method according to claim 11, wherein the method is based on spectroscopic signatures comprising one or more molecular vibrational mode information.

14. The method of claim 1, wherein the method is based on the identification of peaks in the spectroscopic signature of the sample at one or more wavenumber regions within 400-4000 cm⁻¹.

15. (canceled)

16. A trained model configured to receive an input comprising a spectroscopic signature of an infrared (IR) spectroscopic analysis performed on a blood sample from a subject and to provide an output representative of whether or not the subject has either adenoma or cancer.

17.-18. (canceled)

19. A method of training a model comprising:
receiving a plurality of data sets representing spectroscopic signatures for a plurality of subjects obtained from an infrared (IR) spectroscopic analysis on blood samples from the subjects;
receiving cancer data and adenoma data indicating for at least some of the subjects whether the subject has cancer or adenoma; and
training the model to determine a probability of whether a patient has cancer or adenoma based on a spectroscopic signature for the patient, wherein
some of the subjects have cancer and some of the subjects have adenoma.

20.-21. (canceled)

22. A method according to claim 19, wherein the training of the model comprises tuning at least one parameter of the model to optimise the area under the ROC curve and/or to provide a desired sensitivity and/or specificity for a determination as to whether a patient has adenoma or cancer.
